(19) United States
(12) Patent Application Publication
(10) Pub. No.: US 2005/0163811 A1
(43) Pub. Date: Jul. 28, 2005

(54) Topical solutions comprising high concentrations of piperidinopyrimidine derivatives and methods of use thereof

(76) Inventors: Richard Lee, Whittier, CA (US); Victoria L. Garzouzi, Richmond, CA (US)

Correspondence Address: CATALYST LAW GROUP, APC, 4330 LA JOLLA VILLAGE DRIVE SUITE 220, SAN DIEGO, CA, 92122, US

(21) Appl. No.: 10/764,005

(22) Filed: Jan. 23, 2004

(51) Int. Cl. A61K 31/519; A61K 9/00; A61K 31/19

(52) U.S. Cl. 424/400; 514/264.11; 514/574; 514/355; 514/474; 514/559

(57) ABSTRACT

This invention relates to a method for achieving a novel solution comprising a high percentage of a piperidinopyrimidine derivative, more particularly minoxidil. The method of the current invention results in a highly effective solution for facilitating hair growth comprising a high concentration of Minoxidil, that is not cosmetically unattractive when applied to a treatment area and that does not cause skin irritation in the treatment area. In another aspect of the invention, said solution comprising high percentage Minoxidil may further comprise co-active ingredients, such as azelaic acid. The present invention also provides a method for stimulating the growth of hair or for preventing hair loss in humans and lower animals.

### BACKGROUND OF THE INVENTION

Alopecia (e.g., male pattern baldness), is primarily a cosmetic problem in humans, stemming from a deficiency of terminal hair; which is the broad diameter colored hair that is readily seen. In the so-called bald person there is a noticeable absence of terminal hair; however, the skin does contain vellus or fine colorless hair which may require microscopic examination to determine its presence. Current treatments for alopecia and other hair growth disorders include those seeking to convert the fine colorless vellus-like hairs into thicker, broader terminal hairs. One such treatment was serendipitously discovered by Dr. Charles A. Chidsey III.

Dr. Charles A. Chidsey, III discloses and claims in U.S. Pat. No. 4,139,619 the use of minoxidil, 6-amino-1,2-dihydro-1-hydroxy-2-imino-4-piperidinopyrimidine, and related 6-amino-4-(substituted amino)-1,2-dihydro-1-hydroxy-2-iminopyrimidines as a means for (a) increasing the rate of growth of terminal hair, and (b) converting vellus-like hair to growth as terminal hair. (Many, but not all of the compounds considered to be “related 6-amino-4-(substituted amino)-1,2-dihydro-1-hydroxy-2-iminopyrimidines,” are described in U.S. Pat. No. 3,461,461). Dr. Chidsey III, teaches a process for making pharmaceutical compositions comprising minoxidil, said compositions including Topical Creams, Ointments and Solutions containing oily, unattractive and often harsh solvents. Furthermore, in U.S. Pat. No. 4,596,812 Dr. Chidsey III and Dr. Guinter Kahn disclose and claim the use of minoxidil as a therapeutic agent to treat alopecia and arrest and reverse male pattern alopecia.

It is well established that the effectiveness of topical Minoxidil and related 6-amino-4-(substituted amino)-1,2-dihydro-1-hydroxy-2-iminopyrimidines, (“Minoxidil Derivatives”) (hereinafter, both minoxidil and Minoxidil Derivatives will be referred to as “Minoxidil”), in treating male pattern baldness is dose dependant, thus it is desirable to deliver a pharmaceutical composition comprising high percentages of Minoxidil. However, Minoxidil has poor solubility in water and ethanol, and thus the dose of Minoxidil in solutions remains relatively low. Thus, the Minoxidil solutions of the prior art comprise a maximum of 5% to 6.5% Minoxidil, depending on the solvent used. As the term is commonly used in the art, a solution is a watery, runny composition, that will either quickly dry or will evaporate once applied. The prior art addresses the Minoxidil solubility problem by making heavy, oily creams and lotions comprising up to 20% Minoxidil. These creams, lotions and ointments generally contain calamine, wool fat and the like to hold the higher percentage of Minoxidil. One of the biggest problems with these heavy, oily creams, ointments and lotions is that they are cosmetically unattractive when applied to the hair.

In both U.S. Pat. No. 4,139,619 and U.S. Pat. No. 4,596,812, Drs. Chidsey III and Kahn disclose a solution wherein the Minoxidil comprises at most 0.5 to 5% of the total solution in a 12% polyethylene glycol base. Thus, Chidsey III's and Kahn's, solutions comprising these ingredients, as well as those currently marketed only contain up to 5% Minoxidil.

Chidsey III further describes pharmaceutical compositions comprising greater than 5% Minoxidil; however, these formulations require more heavy bases like wool fat, calamine, liquid petroleum and the like, thereby forming creams, ointments or lotions comprising up to 20.0% Minoxidil. While the heavy, oily lotions and ointments are capable of maintaining up to 20% Minoxidil, the high percentages of liquid petroleum or wool fat needed to achieve such percentages is cosmetically undesirable. Baldness treatments requiring the addition of a grease to be worn in the hair for a substantial amount of time would appeal to only a small subset of the balding population, if to any at all.

Numerous other formulations comprising Minoxidil have been published, and, as seen with Chidsey III, when the amount of Minoxidil is greater than 5%, these formulations must also resort to using heavy, oily bases, to keep the Minoxidil in solution. Thus, there is a desire in the art to have a highly effective hair growth formulation that does not have the undesirable look, feel and side effects associated with the lotion, cream and ointment formulations currently available and that allows for enhanced penetration of the active ingredient to those areas where the solution is applied.

U.S. Application No. 20020172649 to So, et al. describes a pharmaceutical composition comprising: at least 5% Minoxidil; a water-based or an alcohol-based solvent; and a propylene glycol co-solvent. In the described pharmaceutical composition, the pH of said composition is adjusted to between 1.0 and 7.0 by addition of an acid, thereby solubilizing the Minoxidil into the solvent/co-solvent system. For lotions, the application method solubilizes up to 12% of the Minoxidil. Solutions of the application use a co-solvent system wherein one of said co solvents is propylene glycol. Propylene glycol causes local irritation and hypersensitivity where applied to the skin. (See, Katzung, B. G., Basic and Clinical Pharmacology McGraw/Hill, Eighth Ed. Page 1059; Catanzaro, J. M., et al., Propylene Glycol Dermatitis, J. Amer. Acad. Dermatol, (1991) 20: 90-5; Warshaw, T. G., et al., Studies of Skin Reactions to Propylene Glycol, J. Invest. Dermatol. (1952) 19: 423-9.) Thus, there is a need in the art for pharmaceutical compositions for hair growth comprising high percentages of Minoxidil in a watery runny solution and which will not cause irritation to the skin.

Others have published the use of a pharmaceutical composition comprising Minoxidil used in conjunction with a co-active ingredient as a method for improving upon the rate of hair growth and vellus to terminal hair conversion. The general idea behind these combination therapies is to supplement the Minoxidil dose response curve with an additional hair growing agent (“co-active ingredient”) thereby bypassing the deficiency in said dose response curve caused by Minoxidil's poor solubility in water and alcohol.

U.S. Pat. No. 5,183,817, issued to Bazzano, teaches a combination of Minoxidil and retinoic acid for the treatment of hair loss. Bazzano's comparative studies show that the addition of retinoic acid to pharmaceutical compositions comprising Minoxidil improved the hair growth response, thus accenting the dose response curve of Minoxidil alone. Bazzano discloses lotions and ointments having 0.5% to 10.0% Minoxidil admixed with retinoic acid; however, said lotions and ointments comprise cosmetically unacceptable heavy or oily bases, and up to 50% propylene glycol, a known skin irritant.

A further example, U.S. Pat. No. 5,026,691, issued to Kligman, teaches a combination of Minoxidil and an anti-inflammatory agent (specifically, hydrocortisone) for treating hair loss. Kligman's comparative studies show that the addition of hydrocortisone to pharmaceutical compositions comprising Minoxidil improved the hair growth response, thus accenting the dose response curve of Minoxidil alone. Kligman discloses pharmaceutical compositions made using the methods of Chidsey III (e.g., comprising propylene glycol), a known skin irritant, and comprising both Minoxidil and hydrocortisone.

In U.S. Pat. No. 5,059,606 to Grollier et al, is disclosed a combination of calcium antagonists (which in themselves neither induce nor stimulate hair growth nor slow hair loss) with certain pyrimidine derivatives to induce and stimulate hair growth and to ameliorate the slowing of hair loss. Grollier discloses that the combination of calcium agonist with pyrimidine derivative acts more quickly than does the pyrimidine derivative alone, and thus the combination allows for a greater hair growth effect using low concentrations of the pyrimidine derivative. In the disclosed combinations, Grollier et al use a propylene glycol base, and a maximum 3% Minoxidil concentration.

Thus, there is a need in the art to increase the concentration of Minoxidil in an efficient, penetrating solution to achieve a highly effective dose of Minoxidil without formulating heavy, oily and cosmetically unattractive creams, lotions and/or ointments; and with out relying on the irritating solvents of the prior art. To satisfy this need in the art, it is necessary that a solution of Minoxidil achieve a greater response along the Minoxidil dose-response curve, and is in a thin, watery, runny, liquid solution rather than a cosmetically undesirable heavy, oily cream, lotion or ointment. Additionally, the need in the art requires that the increased concentration of Minoxidil is preferably not included in a solution that also includes skin irritants, such as propylene glycol and other known irritants. There is a further need for thin, watery, runny, liquid solutions with increased concentration of Minoxidil and comprising co-active ingredients for a still greater hair growing effect.

Accordingly, it is an object of the present invention to overcome one or more of the difficulties and deficiencies related to the prior art. These and other objects and features of the present invention will be clear from the following disclosure.

### BRIEF SUMMARY OF THE INVENTION

The current invention provides novel solutions comprising a high percentage of a piperidinopyrimidine derivative, more particularly minoxidil. Using the compositions and methods of the current invention, Applicant has made a highly effective, non-oily solution for facilitating hair growth that comprises a high concentration of Minoxidil, that is not cosmetically unattractive when applied to a treatment area and that does not cause skin irritation in the treatment area. In another aspect of the invention, said solution comprising high percentage Minoxidil may further comprise co-active ingredients, such as azelaic acid.

The present invention also provides a method for stimulating the growth of hair or for preventing hair loss in humans and lower animals. The solutions of the current invention are topically administered to an application situs to increase the rate of terminal hair growth, stimulate the conversion of vellus-like hair to grow as terminal hair and to help prevent the loss of existing terminal hair. The present invention finds application in all mammalian species, including both humans and animals. In humans, the compounds of the subject invention can be applied for example, to the head, pubic area, upper lip, eyebrows, and eyelids. In animals raised for their pelts, e.g. mink, the compounds can be applied over the entire surface of the body to improve the overall pelt for commercial reasons. The process can also be used for cosmetic reasons in animals, e.g. applied to the skin of dogs and cats having bald patches due to mange or other diseases.

### DETAILED DESCRIPTION OF THE INVENTION

By “topical administration”, as used herein, is meant directly laying on, applying to or spreading on outer skin (membrane epidermal tissue) or hair.

By “application situs”, as used herein, is meant a localized area where it is desired that hair growth be stimulated. In humans the application situs can, for example, be on the head, pubic area, upper lip, eyebrows and eyelids. In animals raised for their pelts (for example, mink) the application situs can be over the entire surface of the body to improve the overall pelt for commercial reasons. The present invention can also be used for cosmetic reasons in animals, e.g., application to the skin of dogs or cats having bald patches due to mange or other diseases.

As used herein the term “Minoxidil” means 6-amino-1,2-dihydro-1-hydroxy-2-imino-4-piperidinopyrimidine, in either its free base or hydrochloric acid salt, and also means 6-amino-1,2-dihydro-1-hydroxy-2-iminopyrimidines. These compounds, as well as the methods for synthesizing those compounds, are discussed in detail in the following issued U.S. patents, all of which are incorporated herein by reference: U.S. Pat. No. 3,461,461, Anthony et al., issued Aug. 12, 1969; U.S. Pat. No. 3,382,247, Anthony et al., issued May 7, 1968; U.S. Pat. No. 3,644,364, Anthony, issued Feb. 22, 1972; and U.S. Pat. No. 4,139,619, Chidsey III, issued Feb. 13, 1979.

The Minoxidil compounds which form the pharmaceutically-active component of the present invention are known in the art to stimulate the growth of mammalian hair when applied topically and prevent further hair loss. Compositions of the present invention contain a safe and effective amount of the Minoxidil component; preferably the compositions contain from about 0.01% or more or Minoxidil, more preferably from about 0.01% to about 20%, even more preferably from about 5% to about 20%, and most preferably about 15%, of this component. Of course, the level of active component will vary with the nature and cause of the condition being treated, the surface area available for application, the particular vehicle selected, and the precise application regimen.

As used herein, the term “solution” means a watery liquid preparation of soluble chemicals dissolved in solvents such as water, alcohol, and the like.

As used herein, the term “lotion” means semisolid emulsions that contain fully dissolved or suspended substances for external application.

As used herein, the term “ointment” means semisolid dosage form for topical application to the skin or mucous membranes. Typically, ointments are based on petrolatum and do not contain sufficient water to separate into a second phase at room temperature.

As used herein, the term “co-active ingredient” refers to a wide variety of compounds that are used in combination with Minoxidil to accent the hair growth process. Said co-active ingredients are generally added to Minoxidil solutions by those of ordinary skill in the art, and include, but are not limited to: azelaic acid; retinoic acid; nicotinic esters; anti-inflammatories; calcium; and the like.

As used herein, the term “desired pH” refers to any pH of the Minoxidil solution that does not negatively impact the high percentage of Minoxidil in a solution.

The hair growth stimulant composition of the present invention contains 0.01% by mass or more of the active component Minoxidil, with 0.01% to 20.0% being preferable, 5.0% to 20.0% being more preferable, 12.5% to 15% being even more preferable and 15% being most preferable.

Components and methods for making the vehicle of the present invention significantly increase the percentage of Minoxidil that will remain soluble in a solution. As the solvent for dissolving the Minoxidil, said vehicle preferably comprises the trihydric alcohol glycerin. Glycerin is a base used to make hand soap that is a neutral, colorless liquid which freezes to a gummy paste and which has a high boiling point. Glycerin is far less irritating and much less of a sensitizing agent that is propylene glycol. Glycerin dissolves into water or alcohol, but not oils. In the preferred embodiment, the glycerin-Minoxidil mixture is dissolved in a lower alcohol, and more preferably in ethyl alcohol. In addition, many things will dissolve into glycerin easier than they do into water or alcohol, thus glycerin forms the primary solvent of the current invention vehicle, while ethyl alcohol forms the secondary solvent. Glycerin is incorporated in an amount according to the desired range of Minoxidil, with from about 10% to about 20% glycerin being preferable and from about 15% to about 20% being particularly preferable. It is also desirable to add ethanol as a second solvent to the above solvent vehicle. The final composition preferably contains ethanol in an amount of from about 80% or less, depending on the volume of components such as the Minoxidil, the specific acid used for pH adjustments and any optional co-active ingredients such as retinoic acid; nicotinic esters; anti-inflammatories; calcium; azelaic acid; or the like. Although the preferred primary solvent is glycerin, Applicant's methods are also applicable to other solvents including propylene glycol, although the use of such a solvent will result in a watery, runny solution comprising a high percentage of Minoxidil and that is a skin irritant.

In a preferred embodiment of the current invention, the vehicle comprises glycerin, ethyl alcohol and the active ingredient (e.g., Minoxidil). In this embodiment, 0.01% to 20.0% Minoxidil is solubilized in glycerin and ethyl alcohol resulting in a watery solution.

The hair growth stimulant composition of the present invention is preferably prepared by first heating glycerin to a range of between from about 55.deg.C. to about 75.deg.C., and preferably heating said glycerin to about 60.deg.C. Following heating, 0.01% or more of the active component Minoxidil, preferably about 6.5% to about 20% and most preferably about 15% of micronized Minoxidil, is added to the heated glycerin solvent. The heated glycerin and micronized Minoxidil is then mixed and whisked for approximately 10 minutes, or for a sufficient time to obtain a homogenous white slurry. Applicant has discovered that the step of heating the glycerin is useful for solubilizing a higher percentage of Minoxidil into a water like solution than can be achieved using the methods of the prior art.

The heated glycerin/Minoxidil solution is brought to a desired volume by rapidly mixing in a secondary solvent alcohol, preferably a lower alcohol, and more preferably 200 proof ethyl alcohol, and distilled water. While constantly and vigorously stirring, the glycerin/Minoxidil/alcohol/water mixture is heated to a range of between from about 35.deg.C. to about 40.deg.C., and preferably to about 40.deg.C. The glycerin/Minoxidil/alcohol/water solution, which is initially an opaque white solution, will begin to become transparent at about 30 deg.C. and will become totally clear at 40 deg.C. A desired pH is achieved. For example, the pH is adjusted to a range of between from about 3.5 to about 6.5, preferably from about 4.5 to about 6.3, and most preferably to a pH of about 5.7+/−0.3 using any of a number of well known, non-irritating pH adjustors, preferably ascorbic acid, citric acid, hydrochloric acid, lactic acid, phosphoric acid, and the like. When preparing the high concentration Minoxidil solution using this formulation method, the solution of Minoxidil will be a clear or slightly amber and water like colored liquid.

In an alternative embodiment of the current invention, the vehicle comprises glycerin, ethyl alcohol, the active ingredient (e.g., Minoxidil) and a co-active ingredient (e.g., Azelaic Acid). In this embodiment, from about 0.01% to about 20.0% of Minoxidil and from about 0.01% to about 5.0% Azelaic acid are solubilized in the glycerin. Those of ordinary skill in the art will readily substitute in place of the Azelaic Acid numerous other co-active ingredients, including, but not limited to retinoic acid; nicotinic esters; anti-inflammatories; and calcium, without undue experimentation. Such substitutions are well within the spirit of the current invention.

The hair growth stimulant composition of the present invention is preferably prepared by first heating glycerin to a range of between from about 55.deg.C. to about 75.deg.C., and preferably heating said glycerin to about 60.deg.C. Following heating, from about 0.01% or more of the active component Minoxidil, preferably about 6.5% to about 20% and most preferably about 15% of Minoxidil, is added to the heated glycerin solvent. The heated glycerin and Minoxidil is then mixed and whisked for approximately 10 minutes, or for a sufficient time to obtain a homogenous white slurry.

Once a homogenous white slurry is achieved the solution is combined with a co-active ingredient, and is brought to volume using a secondary solvent alcohol, distilled water and a pH adjustor. In a preferred embodiment of this current method, a volume of alcohol, preferably ethyl alcohol 200 proof, and between from about 0.01% and about 5.0% Azelaic acid is added to the glycerin/Minoxidil opaque, white slurry. Distilled water, is added to bring the final solution to a desired volume. The combined mixture is heated to from about 35.deg.C. to about 40.deg.C., preferably about 40.deg.C., and stirred continuously until a homogenous and clear mixture is achieved. A desired pH is achieved. For example, the pH is adjusted to a range between from about 3.5 to about 6.5, preferably from about 4.5 to about 6.3 and most preferably to about 5.7+/−0.3. When preparing the high concentration Minoxidil preparation using this formulation method, the solution of Minoxidil will be a clear or slightly amber colored watery liquid. Applicant has discovered through experimentation that when the final solution of Minoxidil and Azelaic acid is brought above 40.deg.C., the final solution becomes tackier, and thus begins to take on cosmetically undesirable properties. Such is true particularly when azelaic acid is used as the co-active ingredient and pH adjustor.

The methods of Applicant's current invention can be similarly used to make solutions comprising high concentration Minoxidil and other co-active ingredients. By way of example only, using the above methods Applicant has made a 15% Minoxidil solution that further comprises ascorbic acid or phosphoric acid in place of the azelaic acid. In such an example, Applicant added a high percentage of Minoxidil to heated glycerin. Once this slurry was homogenized, Applicant added the Ascorbic acid of Phosphoric acid; brought up the volume of the solution and adjusted the pH. Those of ordinary skill in the art will readily make the necessary alterations of the current description to include numerous other co-active ingredients without departing from the spirit of the current invention.

A further variation anticipated by the current invention is the use of a solvent other than glycerin. Although glycerin is preferred because, among other reasons, is formulates a final solution that is cosmetically attractive, holds a high percentage of Minoxidil in solution and is not a general skin irritant, Applicant has used the methods of the current invention with other solvents for bringing a high concentration of Minoxidil in to solution. By way of example only, Applicant heated propylene glycol and added a high concentration of Minoxidil (up to 20%) to the heated propylene glycol. The final solution using this alternative solvent comprised a high concentration of Minoxidil, optionally a co-active ingredient, and was a cosmetically attractive solution; however, said final solution was also a general skin irritant, thusly not addressing all of the stated problems of the prior art. The advantage to showing Applicant's method is useful with propylene glycol is that those who have used this solvent in the prior art, can use Applicant's inventive method and make cosmetically attractive solutions comprising much higher percentages of Minoxidil without changing over their solvent system. As stated; however, said solutions will retain the skin irritant properties of the prior art.

It is notable that one of ordinary skill in the art will readily perform the methods of the current disclosure in a different order and/or otherwise varying the techniques.

The hair growth stimulant composition of the present invention thus obtained can be used as a suitable topical preparation, preferably as a watery solution; however, one of ordinary skill in the art will readily use Applicants' inventive method to prepare lotions, ointments, aerosols, tonics, creams, gels, and the like.

Method of Use

It will be appreciated that this invention provides a method for stimulating the growth of hair in humans and lower animals. In addition, the compositions of the present invention may be applied to hairy areas to prevent hair loss. The present invention permits the significantly improved topical application of the Minoxidil actives defined herein in an aesthetically acceptable, skin substantive composition, without irritating the skin at the site of application.

Topical treatment regimens according to the practice of this invention comprise applying the compositions herein directly to the skin, i.e., at the application situs, usually one to six times daily. The rate of application and duration of treatment will, of course, depend on many factors. A typical safe and effective usage rate for topical treatment is from about 1 ml to about 10 ml of the total topical composition per square centimeter of skin per application. The skilled artisan will appreciate that this application rate will vary with the desired effect, the condition being treated and its cause, its progress and response, the area involved, the severity and nature of the condition being treated, the precise identity of the Minoxidil and or carriers being used, the presence or absence of penetration-interfering solvents, cosolvents, excipients and lipids, the physical condition of the patient, concurrent therapies being administered, the concentration of the actives or carriers being used, as well as other factors within the particular knowledge of the patient and/or physician within the scope of sound medical judgment. Generally, the compositions of the present invention will be used such that a total of from about 2.5 mg to about 100 mg of Minoxidil will be applied each day.

The compositions can be applied from once every twenty-four hours to once every hour. Application intervals of every 4 hours to every 12 hours are preferred. A treatment regimen of application every 12 hours is particularly preferred because it minimizes the amount of Minoxidil which is applied at any one time while reducing the inconvenience of frequent applications. However, any treatment regimen, which allows a safe and effective amount of Minoxidil to reach the afflicted situs can be employed while using the compositions of this invention.

### EXAMPLES

The present invention will be described in more detail by the following examples, which should not be construed as limiting the present invention.

### Example 1

### 100 ml Solution of 15% Minoxidil

In a first example of the current invention, 100 ml of a 15% Minoxidil solution is prepared using the methods of the current disclosure. Although the steps are described linearly, those of ordinary skill in the art will readily vary the order thereof without exceeding the scope of the current disclosure.

In a glass beaker, 20 ml of glycerin (e.g., Glycerin USP, Cat. No.: G2289, Sigma, St. Louis, Mo.) is heated to 60.deg.C. and maintained at said temperature. 150 grams of micronized Minoxidil (e.g., Minoxidil, Powder, USP, Cat. No.: 8518HP, Voigt Global Distribution, Kansas City, Mo.) is added to the heated glycerin and the mixture is stirred and whisked for about 10 minutes or until a homogenous white slurry is obtained. 70 ml of 200 proof ethyl alcohol is added to the Minoxidil/glycerin slurry and heated to between 30.deg.C. and 40.deg.C., while constantly and vigorously stirring. A desired pH is achieved. In this example, the pH is brought to and maintained at between about 3.5 and 6.5, preferably between about 4.5 and about 6.3, most preferably to 5.7+/−0.3 using 35 mg citric acid, (e.g., Citric Acid, Anhydrous, Granular, USP, Cat. No.: C1133, Voigt Global Distribution, Kansas City, Mo.). The solution is brought to the final desired volume of 100 ml using deionized water (approximately 10 ml). The final product is a clear 15% Minoxidil solution that is neither a skin irritant, nor a heavy, greasy and cosmetically unattractive lotion or ointment.

### Example 2

### 100 ml Solution of 15% Minoxidil and 5% Azelaic Acid

In a second example of the current invention, 100 ml of a 15% Minoxidil and 5% Azelaic Acid solution is prepared using the methods of the current disclosure. Although the steps are described linearly, those of ordinary skill in the art will readily vary the order thereof without exceeding the scope of the current disclosure.

In a glass beaker, 20 ml of glycerin (Cat. No.: G2289, Sigma) is heated to 60.deg.C. and maintained at said temperature. 150 grams of micronized Minoxidil (Cat. No.: 8518HP, Voigt Global Distribution) is added to the heated glycerin and the mixture is stirred and whisked for about 10 minutes or until a homogenous white slurry is obtained. 70 ml of 200 proof ethyl alcohol and 50 grams of Azelaic Acid are added to the Minoxidil/glycerin slurry and heated to between 30.deg.C. and 40.deg.C., while constantly and vigorously stirring. The solution is brought to the final desired volume of 100 ml using deionized water (approximately 10 ml). The final product is a clear 15% Minoxidil/5% Azelaic Acid Solution that is neither a skin irritant, nor a heavy, greasy and cosmetically unattractive lotion or ointment.

### Example 3

### 100 ml Solution of 20% Minoxidil

In a third example of the current invention, 100 ml of a 20% Minoxidil solution is prepared using the methods of the current disclosure. Although the steps are described linearly, those of ordinary skill in the art will readily vary the order thereof without exceeding the scope of the current disclosure.

In a glass beaker, 20 ml of glycerin (e.g., Glycerine USP, Cat. No.: G2289, Sigma, St. Louis, Mo.) is heated to 60.deg.C. and maintained at said temperature. 200 grams of micronized Minoxidil (e.g., Minoxidil, Powder, USP, Cat. No.: 8518HP, Voigt Global Distribution, Kansas City, Mo.) is added to the heated glycerin and the mixture is stirred and whisked for about 10 minutes or until a homogenous white slurry is obtained. 70 ml of 200 proof ethyl alcohol is added to the Minoxidil/glycerin slurry and heated to between 30.deg.C. and 40.deg.C., while constantly and vigorously stirring. A desired pH is achieved. In this example, the pH is brought to about 3.5 and 6.5, preferably to about 4.5 and about 6.3, most preferably to 5.7+/−0.3 using 35 mg citric acid, (e.g., Citric Acid, Anhydrous, Granular, USP, Cat. No.: C1133, Voigt Global Distribution, Kansas City, Mo.). The solution is brought to the final desired volume of 100 ml using deionized water (approximately 10 ml). The final product is a clear 20% Minoxidil solution that is neither a skin irritant, nor a heavy, greasy and cosmetically unattractive lotion or ointment.

### Example 4

### 12.5% Minoxidil in Propylene Glycol and Optionally Comprising a co-Active Ingredient

In a fourth example, the current invention for bringing a high percentage of Minoxidil into solution is described using a propylene glycol solvent. Although propylene glycol is not the preferred solvent for the reasons stated above, Applicants have applied their invention to such a solvent in order to teach a cosmetically attractive solution comprising a high percentage of Minoxidil without requiring those in the art using said solvent to change their solvent system.

Formula:

Phase A: 40.00(v) % w/v Propylene Glycol USP. Phase B: 12.50(w) % w/v Minoxidil USP micronized. Phase C, 39.80(v) % w/v SD Alcohol 40_B 200 proof; 20.00(v) % w/v Benzyl Benzoate USP; 0.20(v) % w/v Benzyl Nicotinate; (optionally) 5.00(w) % w/v Azelaic Acid.

Manufacture:

Phase A was heated to 65.deg.C. and Phase B added to the heated Phase A with good agitation. Agitation continued for approximately 10 minutes or until mixture became homogenous with an opaque white look. Agitation continued as the mixture was allowed to cool to about 45.deg.C. The volume of the mixture was brought up by adding Phase C, which may optionally contain a co-active ingredient (e.g., Azelaic Acid). Mixing was continued until the solution homogenized and a clear, watery solution formed. The final product comprised 12.5% Minoxidil, and optionally comprised Azelaic Acid in a watery, clear solution that was not cosmetically unattractive.

The compositions described in the above examples, as well as other compositions made by those of ordinary skill in the art using the teachings of the current invention are substantial improvements over the current art. The teachings of the current invention allow those of ordinary skill to prepare high dose Minoxidil solutions, which in turn have an improved hair growth response. The high dose of Minoxidil is solubilized in a non-greasy solvent, and thus is not cosmetically unattractive when used in the hair. Furthermore, the solvent of the current invention is not a skin irritant.

1. A method for making a topical solution comprising the steps of:
heating a solvent to a temperature of between about 55.deg.Celsius and about 75.deg.Celsius;
adding a piperidinopyrimidine derivative to said heated solvent;
thoroughly mixing said heated solvent and said piperidinopyrimidine derivative until a homogenous white slurry is achieved;
mixing in an appropriate amount of an alcohol to said solvent and piperidinopyrimidine derivative mixture;
heating said alcohol, solvent, water and piperidinopyrimidine derivative to a temperature of between about 30.deg.Celsius and about 40.deg.Celsius; and
having a desired pH of said alcohol, solvent and piperidinopyrimidine derivative mixture.

2. The method of claim 1, wherein said solvent is glycerin.

3. The method of claim 1, wherein said solvent is heated to a temperature of about 60.deg.Celsius.

4. The method of claim 1, wherein said piperidinopyrimidine derivative is a 6-amino-1,2-dihydro-1-hydroxy-2-iminopyrimidine.

5. The method of claim 1, wherein said piperidinopyrimidine derivative is 6-amino-1,2-dihydro-1-hydroxy-2-imino-4-piperidinopyrimidine.

6. The method of claim 1, wherein the concentration of said piperidinopyrimidine derivative is 0.01 to 20.0 percent of the topical solution.

7. The method of claim 1, wherein the concentration of said piperidinopyrimidine derivative is 7.5 to 20.0 percent of the topical solution.

8. The method of claim 1, wherein the concentration of said piperidinopyrimidine derivative is 7.5 to 15.0 percent of the topical solution.

9. The method of claim 1, wherein the concentration of said piperidinopyrimidine derivative is 15 percent of the topical solution.

10. The method of claim 1, wherein said alcohol is ethyl alcohol.

11. A topical solution made from the method recited in claim 1.

12. A method for making a topical solution comprising the steps of:
heating a solvent to a temperature of between about 55.deg.Celsius and about 75.deg.Celsius;
adding a piperidinopyrimidine derivative to said heated solvent to make a first mixture;
thoroughly mixing said first mixture until a homogenous white slurry is achieved;
adding a co-active ingredient to an alcohol to make a second mixture;
heating said second mixture to a temperature of between about 30.deg.Celsius and about 40.deg.Celsius; and
having a desired pH reading for said third mixture.

13. The method of claim 12, wherein said solvent is glycerin

14. The method of claim 12, wherein said solvent is heated to a temperature of about 60.deg.Celsius.

15. The method of claim 12, wherein said piperidinopyrimidine derivative is a 6-amino-1,2-dihydro-1-hydroxy-2-iminopyrimidine.

16. The method of claim 12, wherein said piperidinopyrimidine derivative is 6-amino-1,2-dihydro-1-hydroxy-2-imino-4-piperidinopyrimidine.

17. The method of claim 12, wherein the concentration of said piperidinopyrimidine derivative is 0.01 to 20.0 percent of the topical solution.

18. The method of claim 12, wherein the concentration of said piperidinopyrimidine derivative is 7.5 to 20.0 percent of the topical solution.

19. The method of claim 12, wherein the concentration of said piperidinopyrimidine derivative is 7.5 to 15.0 percent of the topical solution.

20. The method of claim 12, wherein the concentration of said piperidinopyrimidine derivative is 15 percent of the topical solution.

21. The method of claim 12, wherein said co-active ingredient is selected from the group comprising: azelaic acid; ascorbic acid; retinoic acid; nicotinic esters; anti-inflammatories; and calcium.

22. The method of claim 21, wherein said co-active ingredient is azelaic acid.

23. The method of claim 12, wherein said alcohol is ethyl alcohol.

24. A topical solution made from the method recited in claim 12.

25. A topical solution for application to mammalian skin comprising at least an effective amount of a piperidinopyrimidine derivative dissolved in a solvent wherein said solution comprises from about 0.01 percent to about 20.0 percent piperidinopyrimidine derivative.

26. The topical solution of claim 25, wherein said solution comprises about 7.5 percent to about 20.0 percent piperidinopyrimidine derivative.

27. The topical solution of claim 25, wherein said solution comprises about 15.0 percent piperidinopyrimidine derivative.

28. The topical solution of claim 25, wherein said piperidinopyrimidine derivative is a 6-amino-1,2-dihydro-1-hydroxy-2-iminopyrimidine.

29. The topical solution of claim 25, wherein said piperidinopyrimidine derivative is 6-amino-1,2-dihydro-1-hydroxy-2-imino-4-piperidinopyrimidine.

30. The topical solution of claim 25, further comprising a lower alcohol as a secondary solvent.

31. The topical solution of claim 30, wherein said lower alcohol is ethyl alcohol.

32. The topical solution of claim 25, further comprising a co-active ingredient selected from the group consisting of azelaic acid; ascorbic acid; retinoic acid; nicotinic esters; anti-inflammatories; and calcium.

33. The topical solution of claim 32, wherein said co-active ingredient is azelaic acid.

34. The topical solution of claim 25, wherein said solvent is a trihydric alcohol.

35. The topical solution of claim 34 wherein said solvent is glycerin.

36. The topical solution of claim 25, wherein said solvent is a dihydric alcohol.

37. The topical solution of claim 36, wherein said solvent is propylene glycol.

38. A method for stimulating hair growth, which comprises topically applying to the area where hair growth is desired an effective amount of a pharmaceutical composition at least comprising at least 6.5 percent of a piperidinopyrimidine derivative in a solution.

39. The method of claim 38, wherein said piperidinopyrimidine derivative is a 6-amino-1,2-dihydro-1-hydroxy-2-iminopyrimidine.

40. The method of claim 38, wherein said piperidinopyrimidine derivative is 6-amino-1,2-dihydro-1-hydroxy-2-imino-4-piperidinopyrimidine.

41. The method of claim 38, wherein said pharmaceutical composition further comprises a compound selected from the group comprising: azelaic acid; ascorbic acid; retinoic acid; nicotinic esters; anti-inflammatories; and calcium.

42. The method of claim 41, wherein said co-active ingredient is azelaic acid.

43. The method of claim 38, further comprising a solvent that is a trihydric alcohol.

44. The method of claim 43, wherein said solvent is glycerin.

45. The method of claim 38, further comprising a solvent that is a dihydric alcohol.

46. The method of claim 45, wherein said solvent is propylene glycol.
